(19) United States
(12) Patent Application Publication
(10) Pub. No.: US 2009/0187250 A1
(43) Pub. Date: Jul. 23, 2009

(54) JOINT

(75) Inventor: Hans Naegerl, Gleichen (DE)

Correspondence Address: DARBY & DARBY P.C., P.O. BOX 770, Church Street Station, New York, NY, 10008-0770, US

(73) Assignee: AEQUOS Endoprothetik GmbH

(21) Appl. No.: 12/049,842

(22) Filed: Mar. 17, 2008

(30) Foreign Application Priority Data
Mar. 15, 2007 (DE) 10 2007 013 121.8

(51) Int. Cl. A61F 2/38; A61F 2/30

(52) U.S. Cl. 623/20.14; 623/18.11

(57) ABSTRACT

An artificial joint as an endoprosthesis for a human joint, has at least two joint parts wherein each joint part has two functional surfaces that operatively interact with each other, one associated with the first joint part and the other with the second joint part. The two functional surfaces of each joint part are spheroidal in shape and convex-concave, concave-convex or convex-convex in shape in the proximal-distal arrangement, and the functional surfaces can each pivot around a pivoting axis. In order to create an artificial joint as an endoprosthesis for a human joint, whose motion behavior is perceptibly improved for the patient, the two pivoting axes of the two functional surfaces of each joint part are slanted towards each other.

Priority is claimed to German Patent Application No. DE 10 2007 013 121.8, filed Mar. 15, 2007, the entire disclosure of which is incorporated by reference herein.

The present invention relates to an artificial joint as an endoprosthesis for a human joint, consisting of at least two joint parts, whereby each joint part has two functional surfaces that operatively interact with each other, one associated with the first joint part and the other with the second joint part, whereby the two functional surfaces of each joint part are spheroidal in shape and convex-concave, concave-convex or convex-convex in shape in the proximal-distal arrangement, and the functional surfaces can each pivot around a pivoting axis. In particular, the invention relates to such an endoprosthesis for the human knee-joint that has functional surfaces associated with the femur on the one hand and with the tibia on the other hand, whereby the two functional surfaces of each joint part are spheroidal in shape and convex-concave, concave-convex or convex-convex in shape in the femoral-tibial arrangement.

### BACKGROUND

A joint of the generic type is disclosed, for example, in European patent application EP 07 34 700 A2, in which the joint geometry of the functional surfaces with respect to each other in each of the two planes is defined by a joint chain having two joint axes that run through the rotation centers of the functional surfaces with the radii of the corresponding intersection contours, whereby a connection on the femur side, i.e. on the condyle side, of the mid-points of the condyles corresponds to a frame, and a connection on the tibia side, i.e. on the socket side, of the mid-points of the sockets corresponds to a connecting link of a four-joint chain having the four axes.

German patent application DE 196 46 891 A1 discloses an artificial joint, especially an endoprosthesis to replace natural joints, consisting of at least two artificial joint parts having curved articulation surfaces, a curved contact line being formed on each articulation surface. The curved contact line of one of the articulation surfaces is part of an elliptical intersection contour of a first cylinder or cone having the cylinder radius or cone angle. The other contact line takes the form of a counter track of a second cylinder or second cone that rolls or slides on the first cylinder or on the first cone together having the cylinder radius or the cone angle. The articulation surfaces have regulating surfaces formed from a plurality of straight touching lines. These regulating surfaces—opposite from each other—adjoin the contact lines, and the touching lines each constitute the connection lines between a momentary point of contact of the contact lines and a momentary shared point or the momentary pole of the appertaining motion systems in a reference plane or a reference cone in the moved/unmoved system.

German patent application DE 195 21 597 A1 relates to an artificial joint, especially to an endoprosthesis to replace natural joints, consisting of at least two artificial joint parts having curved articulation surfaces, whereby a circular-arc contact line is formed on each of the articulation surfaces, said contact line being a partial section of a contact circle that lies in a plane and has a mid-point. The articulation surfaces are arranged as a pair in such a way with respect to each other that the contact lines can roll on each other, and axes that run perpendicular to the plane of the contact circles and that run through the mid-point thereof intersect each other in a point of intersection. Regulating surfaces made up of a plurality of straight touching lines are formed on one side of the contact lines, whereby the touching lines lie on momentary lines that connect the momentary contact points that are created during the rolling motion to the momentary point of intersections that result from a pivoting motion of the contact lines at an angular velocity around a shared tangent of the contact lines through the momentary contact points.

European patent application EP 600 806 A1 already discloses a three-part knee prosthesis with a femoral and a tibial implant, whereby the femoral implant has a notch defining two diverging condyle saddles connected by a trochlea are circumscribed, whereby the femoral implant has a shape resulting from the combination of the following features, namely, that the inner and the outer condyle saddles have different curvature radii in the sagittal plane, that the bearing and sliding surfaces of the inner and outer condyle saddles have a different width and cross section in the frontal plane, that the inner and outer condyle saddles have different rolling amplitudes in the posterior part, that the trochlea delimits a surface having the cross section of a geometrical torus in the frontal and sagittal planes, whereby they are raised anatomically on the outer side, that the extension of the support and sliding surfaces of the condyle saddles is raised relative to the anatomical circumstances and the condyle saddles are flattened in order to enhance the pressure distribution.

German patent application DE 39 08 958 A1 already discloses an artificial joint intended as a replacement for human joints, said artificial joint consisting of two joint parts having movable functional surfaces. The curvature ratios of the functional surfaces that have a circular intersection contour are convex-convex, convex-concave or concave-concave with respect to each other, and the joint geometry is defined by a joint chain having two joint axes (dimeric joint chain) that run through the rotation centers of the functional surfaces. In this context, the joint surfaces have a spherical configuration so as to permit joint motion with five degrees of freedom.

### SUMMARY OF THE INVENTION

An aspect of the present invention is to provide an artificial joint as an endoprosthesis for a human joint, whose motion behavior is perceptibly improved for the patient.

The present invention provides an artificial joint for the human knee-joint, in which both pivoting axes of the two functional surfaces of each joint part are slanted towards each other. As a result, it is achieved with surprisingly simple means that, when the knee-joint is bent, a path of motion is created that does not run, for example, in the sagittal plane, but rather, that corresponds to a three-dimensional path of motion. In other words, the slant of the pivoting axes gives rise to a path leading laterally out of the main functional plane, so that, during walking, the foot is guided along a laterally arcuate path of motion relative to the plane of the ground surface. In this process, the position of the momentary twisting or rotational axis is dependent on the flexion angle of the knee-joint. A multiple joint formed by the two pivoting axes of the tibial functional surfaces and the two pivoting axes of the corresponding femoral functional surfaces prescribes these positions of the momentary twisting or rotational axis as a function of the flexion angle.

The two pivoting axes of the functional surfaces of a joint can each have a point of intersection. An especially advantageous embodiment of the present invention is also attained in that the four pivoting axes of the two functional surfaces intersect each other at a shared point of intersection. As a result, a reliable rolling motion defined by the flexion angle is achieved for the two functional surfaces without their detrimentally affecting each other, thus especially preventing a forcible sliding of one of the two functional surface pairs or even an interruption of the contact between the functional surfaces of a joint part. Of course, the desired sliding motion of the functional surfaces in a prescribed flexion range of the knee-joint can be adjusted without any limitations.

In contrast, it has also been proven to be particularly practical for the shared point of intersection of the pivoting axes of the functional surfaces of the two joint parts to be situated on the medial side, resulting in a path of motion, for example, of the tibia, along a path of motion having a convex shape in the lateral direction. To put it in simple terms, a pendulum-like motion of the lower leg of the patient around her/his standing leg is superimposed onto the flexion motion during walking or running, whereby the sequence of motions of the patient thus achieved is felt to be considerably more pleasant. The shared point of intersection of the pivoting axes of the functional surfaces of the two joint parts, for example, in an application as an artificial thumb joint, can be arranged radially.

Another likewise particularly advantageous embodiment of the joint according to the invention is achieved in that the functional surfaces are configured as a rotational body relative to their appertaining pivoting axis. For this purpose, the rotation-symmetrical body can be configured, for instance, to be spindle-shaped or barrel-shaped or else to have a spheroidal, for example, elliptical or toric cross section in the longitudinal extension.

In contrast, a likewise especially practical modification of the present invention is achieved in that the functional surfaces are configured to have a truncated-conical shape in certain sections so as to allow a line contact of the functional surfaces of a joint part parallel to the pivoting axis. For example, this line contact could be limited to certain flexion angles of the knee-joint where especially high states of load occur.

It has also been found to be particularly practical if the functional surfaces either roll or slide on each other as a function of the flexion angle of the joint, or else the ratio of the rolling and sliding is varied as a function of the flexion angle. This results in a combination of a rolling motion in certain sections as well as a sliding motion that, in an optimal manner, approximates the requirements of the joint to duplicate the natural sequence of motions.

When the artificial joint is employed as an endoprosthesis for a human knee-joint, the pivoting axis of the medial functional surface of the femoral joint part in the area of the joint is offset in the anterior direction, in the anterior caudal direction or in the anterior cranial direction relative to the pivoting axis of the lateral functional surface of the femoral joint part.

### BRIEF DESCRIPTION OF THE DRAWINGS

The invention allows for different configurations. In order to further elucidate its basic principle, one of these is depicted in the drawing and will be described below. The drawing schematically shows the following:

FIG. 1—a perspective view of a joint according to the invention;

FIG. 2—a cutaway view of a lateral joint part of the joint shown in FIG. 1, in a sectional view;

FIG. 3—a cutaway view of a medial joint part of the joint shown in FIG. 1, in a sectional view.

### DETAILED DESCRIPTION

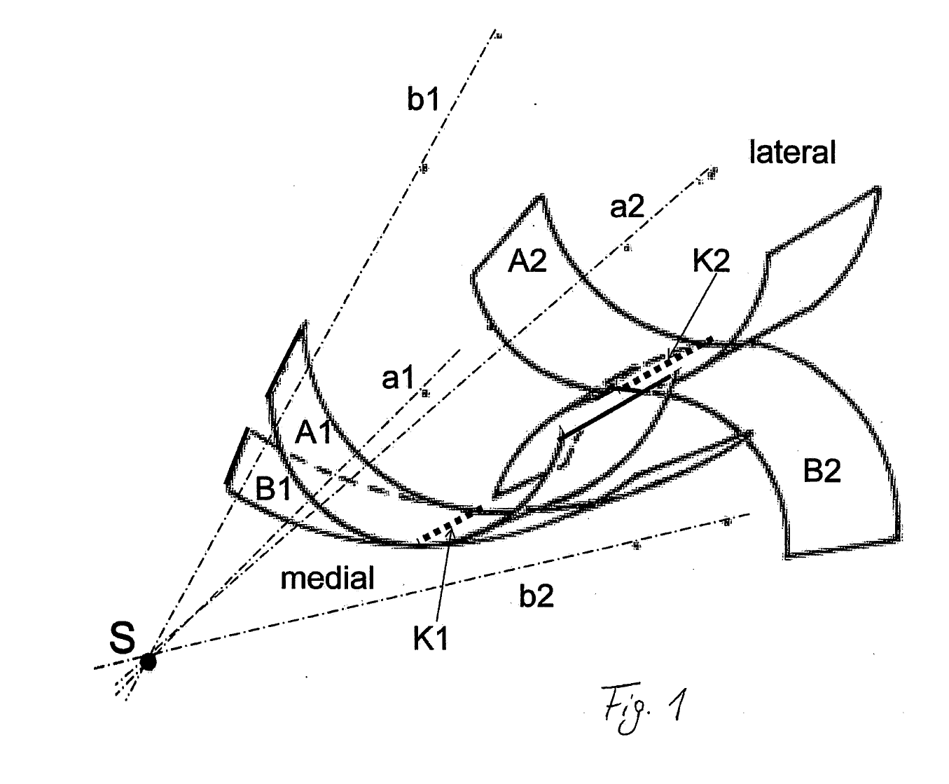

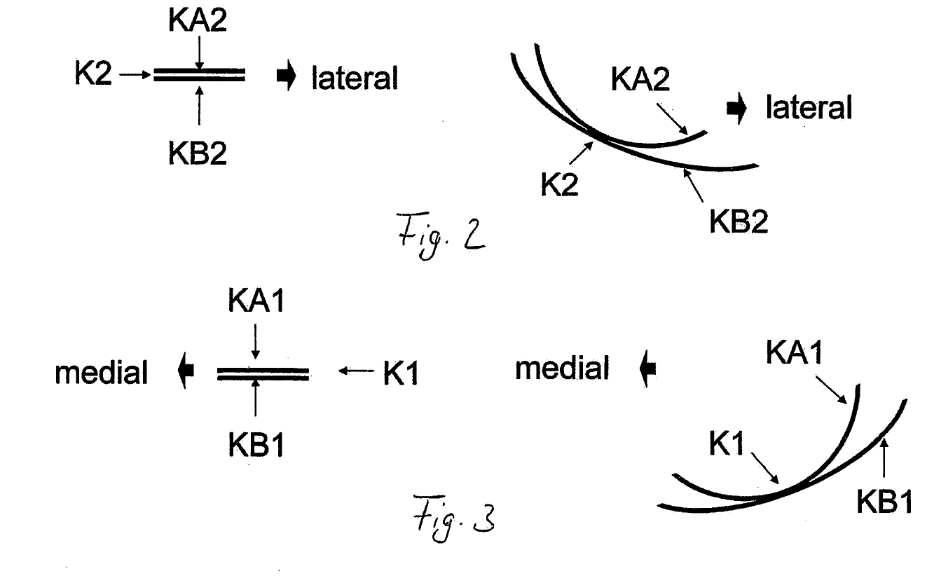

An artificial joint according to the invention as an endoprosthesis for a human joint will be described in greater detail on the basis of FIGS. 1 to 3, which show the joint in a perspective view and, in FIGS. 2 and 3, in a cutaway view of a lateral joint part A and of a medial joint part B. Each joint part A, B has two spheroidal functional surfaces A1, A2, B1, B2 that operatively interact with each other, each associated with the femoral joint part on the one hand, and with the tibial joint part on the other hand. In the proximal-distal arrangement, the functional surfaces B1, B2 of the medial joint part B are concave-convex and the functional surfaces A1, A2 of the lateral joint part A are convex-convex. The appertaining pivoting axes a1, a2, b1, b2 of the functional surfaces A1, A2, B1, B2 of each joint part A, B are slanted towards each other and intersect each other at a shared point of intersection S. The shared point of intersection S of the pivoting axes a1, a2, b1, b2 of the functional surfaces A1, A2, B1, B2 is situated on the medial side outside of the joint.

The contact K1, K2 formed between the functional surfaces A1, A2, B1, B2 is depicted in greater detail in FIGS. 2 and 3, which show a view of the functional surfaces B1, B2 along the line K1 and K2. Each of the contact areas KA1, KA2 as well as KA2, KB2 are visible as a convex-concave arrangement of the functional surfaces A1, A2, B1, B2, which are only shown in sections. In this context, the especially punctiform contact K1, K2 is not formed in the lowest point of the contact areas KB1 and KB2, but rather, in an area having a moderate incline in an area facing each of the other functional surfaces A1, A2, B1, B2 of the same joint part A, B.

What is claimed is:

1. An artificial joint as an endoprosthesis for a human joint, comprising:
a first joint part having a first functional surface pivotable about a first pivoting axis and a second functional surface pivotable about a second pivoting axis;
a second joint part having a third functional surface pivotable about a third pivoting axis and a fourth functional surface pivotable about a fourth pivoting axis, wherein the first functional surface operatively interacts with the third functional surface and the second functional surface operatively interacts with the fourth functional surface, wherein each of the functional surfaces are spheroidal in shape and interact in one of a convex-concave, concave-convex and convex-convex manner in a proximal-distal direction, wherein the first, second, third and fourth axes are slanted towards each other and intersect each other at a common point of intersection.

2. The artificial joint as recited in claim 1, wherein the shared point of intersection of the pivoting axes is disposed on a medial side of the joint.

3. The artificial joint as recited in claim 1, wherein the shared point of intersection of the pivoting axes is disposed outside of the two joint parts.

4. The artificial joint as recited in claim 1, wherein at least one of the joint parts is a condyle, and wherein pivoting axes of the functional surfaces of the condyles coincide.

5. The artificial joint as recited in claim 1, wherein the first and second functional surfaces includes two rotation-symmetrical bodies.

6. The artificial joint as recited in claim 1, wherein each of the functional surfaces are configured as a rotational body relative to the respective pivoting axis.

7. The artificial joint as recited in claim 1, wherein each of the functional surfaces are configured to have a truncated-conical shape in at least one section.

8. The artificial joint as recited in claim 1, wherein the functional surfaces operatively interact by one of rolling and sliding on each other as a function of a flexion angle of the joint.

9. The artificial joint as recited in claim 8, wherein a ratio of the rolling and sliding is variable as a function of the flexion angle.

10. The artificial joint as recited in claim 1, wherein the human joint is a human knee-joint and wherein the first and second joint parts are configured to at least partially replace a femur and/or a tibia.

11. The artificial joint as recited in claim 1, wherein the human joint is a human knee-joint, wherein the second joint part is a femoral joint part and the first joint part is a tibial joint part, wherein the third and fourth pivoting axes in the area of the joint is offset in one of an anterior direction, an anterior caudal direction and an anterior cranial direction relative to the first and second pivoting axes.

12. The artificial joint as recited in claim 1, wherein the human joint is a human knee-joint, wherein the second joint part is a femoral joint part and the first joint part is a tibial joint part, and wherein a medial functional surface of the femoral joint part has a further pivoting axis.

13. The artificial joint as recited in claim 1, wherein the human joint is a human knee-joint, wherein the second joint part is a femoral joint part and the first joint part is a tibial joint part, and wherein and all of the pivoting axes of the femoral joint part coincide.

14. The artificial joint a as recited in claim 1, wherein the human joint is a human knee-joint, wherein the second joint part is a femoral joint part and the first joint part is a tibial joint part, and wherein the two pivoting axes of the tibial joint part coincide.
